(19) United States
(12) Patent Application Publication
(10) Pub. No.: US 2017/0273887 A1
(43) Pub. Date: Sep. 28, 2017

(54) COMPOSITE POWDER, METHOD OF PRODUCING THEREOF AND COSMETIC COMPOSITION CONTAINING THE SAME

(71) Applicant: TAIR JIUH ENTERPRISE CO., LTD., TAINAN CITY (TW)

(72) Inventors: Po-Han CHEN, TAINAN CITY (TW); Ya-Yun TSAI, TAINAN CITY (TW); I-Ming CHEN, TAINAN CITY (TW); Tung-Liang CHEN, TAINAN CITY (TW)

(21) Appl. No.: 15/448,556

(22) Filed: Mar. 2, 2017

(30) Foreign Application Priority Data
Mar. 22, 2016 (TW) 105108856

(51) Int. Cl. A61K 8/73; A61Q 1/00; A61Q 19/00; A61K 8/06; A61K 8/65

(52) U.S. Cl. CPC A61K 8/736; A61K 8/062; A61K 8/65; A61Q 19/00; A61Q 1/00; A61K 2800/412; A61K 2800/63

(57) ABSTRACT

The present invention relates to a method of producing a composite powder. A porous powder is physically covered by collagen and chitosan sequentially, thereby forming the composite powder. The composite powder has good dispersity, an ability of moisture retention and a low greasiness while being added in an oil-in-water emulsion base or a hydrogel base to form a cosmetic composition.

### RELATED APPLICATIONS

This application claims priority to Taiwan Application Serial Number 105108856, filed Mar. 22, 2016 which is herein incorporated by reference.

### BACKGROUND

Field of invention

The present invention relates to a composite powder and a method of producing the same. More particularly, the present invention relates to a composite powder obtained from physically wrapping a porous powder. The composite powder is added into various aqueous bases, thereby forming a cosmetic composition having good dispersity, high moisture retention and low greasiness.

Description of Related Art

With increasing market requirements of beauty care, various methods for improving convenience, comfortability, process simplicity and performance of cosmetics. The cosmetics such as a foundation cream, a mascara, an eye shadow or a blush in the market includes various powders to improve durability on the skin a luster, a moisture retention or a concealing ability. Generally, the cosmetics is obtained by mixing the powders with compositions such as a bio-compatible molecule (molecules such as gelatin, a seaweed gel, collagen, polysaccharide and the like) other functional composition (e.g. vitamin C, plant extracts and the like) or a pigment. Therefore, the aforementioned powders play an important role in various cosmetics.

Common powders are mainly an organic powder such as an organic silicide, polystyrene, polyethylene terephthalate, nylon, poly(meth)acrylic acid, a derivative of cellulose, or an inorganic powder such as silicone dioxide or titanium dioxide. The hydrophilicity of the powders is insufficient that a chemical modification is usually applied to improve the hydrophilicity and bio-compatibility.

The chemical modification may be, for example, adding a hydrophilic molecule (or bio-compatible molecule) to form a chemical bonding between the powders and the hydrophilic molecule n existence of a cross-linking agent. Besides, the powders may be selectively combined with an oiling agent (e.g. fatty acid having a long chain, an ester, a poly alcohol and the like), to improve dispersity of the powders.

If the powders were not chemically modified but directly used in producing cosmetics, the fitness to the skin of the cosmetics would be not sufficient, and the cosmetics were likely to aggregating due to the sweat and the oil secreted from the human skin. Accordingly, pores are likely to being blocked, leading to malaise. In addition, if the powders were not modified with the hydrophilic functional groups, problems such as insufficient moisture retention, dryness, slacking off after long-term use would occur.

On the other hand, most powders produced by common chemical modification have only a single-layered modification, and the powders are likely to irritate skin due to ionization of the powders by pH or light-exposure. Moreover, even the powders are chemically modified, the dispersity cannot achieve the requirement of the industry due to precipitation of the powders after long-term standing. When adding the powders into the bases of cosmetics, the powders may results in uneven color. Moreover, the chemical cross-linking agent or the oiling agent for improving the dispersity increases greasiness of cosmetics.

Therefore, it is necessary to provide a composite powder to improve drawbacks of the cosmetics, such as insufficient moisture retention, dryness, insufficient dispersity, greasiness and the like.

### SUMMARY

Therefore, an aspect of the present invention provides a method of producing a composite powder, in which, collagen and chitosan are attached to a porous powder through physical adhesion and physical cross-linking, there by forming a double-layered wrap on the porous powder. Dispersity in various bases of the porous powder is improved, moisture retention of the composite powder increases, and greasiness of the composite powder is reduced.

Another aspect of the present invention provides a composite powder, which is produced by the aforementioned method of producing the composite powder.

Another aspect of the present invention provides an oil-in-water cosmetic composition, which includes the aforementioned composite powder.

The other aspect of the present invention provides a hydrogel cosmetic composition, which includes the aforementioned composite powder.

According to the aspects of the present invention, a method of producing a composite powder is provided. In one embodiment, firstly, a first mixing step is performed on a porous powder and collagen in a first solution, so as to form a first mixture solution, in which the collagen has a first weight-average molecular weight of 3 kDa to 30 kDa. Then a first filtering step is performed on the first mixture solution, so as to form a semifinished powder. Next, a second mixing step is performed on chitosan and the aforementioned semifinished powder in a second solution, so as to form a second mixture solution, in which the chitosan has a second weight-average molecular weight of 3 kDa to 300 kDa. Afterwards, a second filtering step is performed on the second mixture solution, thereby forming the composite powder. Based on an amount of the porous powder as 100 parts by weight, an amount of the collagen is 0.1 parts by weight to 20 parts by weight, and an amount of the chitosan is 0.1 parts by weight to 20 parts by weight.

According to one embodiment of the present invention, the porous powder includes an organic powder, an inorganic powder or a combination thereof having a specific surface area of 0.5 m²/g to 200 m²/g and a first average particle size of 2 μm to 20 μm.

According to one embodiment of the present invention, the first solution has a pH of 5 to 9, and the second solution has a pH of 4 to 7.

According to one embodiment of the present invention, weight ratio of the collagen and the first solution is 0.001 to 0.2, and a weight ratio of the chitosan and the second solution is 0.001 to 0.2.

According to one embodiment of the present invention, the first mixing step is performed at a first temperature of 4° C. to 40° C. for 5 minutes to 2 hours.

According to one embodiment of the present invention, the second mixing step is performed at a second temperature of 4° C. to 40° C. for 5 minutes to 2 hours.

According to one embodiment of the present invention, the first solution comprises water, an acetic acid solution or a hydrochloric acid solution, and the second solution comprises water, a lactic acid solution, and an acetic acid solution, a citric acid solution, a mandelic acid solution or a hydrochloric acid solution.

According to the aspects of the present invention, a composite powder is provided by the aforementioned method. In one embodiment, the composite powder includes a porous powder, a collagen layer and a chitosan layer, in which the collagen layer covers an outer surface of the porous powder, and the chitosan layer covers the collagen layer.

According to one embodiment of the present invention, second average particle size of the composite powder 3 μm to 25 μm.

According to the aspects of the present invention an oil-in-water cosmetic composition is provided. The oil-in-water cosmetic composition includes an oil-in-water emulsion base and the aforementioned composite powder that is uniformly dispersed therein.

According to the aspects of the present invention, a hydrogel cosmetic composition is provided. The hydrogel cosmetic composition includes a hydrogel base and the aforementioned composite powder that is uniformly dispersed therein.

When the composite powder and the method of producing the same of the present invention are applied, a semifinished powder is formed by wrapping the porous powder by collagen, followed by rapping the semifinished powder by chitosan, thereby forming the composite powder. The composite powder produced by the aforementioned method has good dispersity, high moisture retention and low greasiness in the oil-in-water cosmetic composition or the hydrogel cosmetic composition.

### DETAILED DESCRIPTION

The present invention provides a method of producing a composite powder, in the method, a porous powder collagen and chitosan are sequentially mixed in solutions having a specific pH, followed by removing a liquid of the solutions, there by forming the composite powder.

That is, in the aforementioned method, the collagen and the chitosan are respectively attached to the porous powder by physical adhesion and physical cross-linking, thereby forming a double-layered wrap on the porous powder. Dispersity of the porous powder in various bases is improved, moisture retention of the porous powder increases, and greasiness is reduced. Therefore, the composite powder produced by the aforementioned method has good dispersity, moisture retention and low greasiness, and thus the composite powder may be applied to various types of cosmetics (e.g. skin care products or makeup).

The physical adhesion of the present invention refers to physically wrapping a surface of the porous powder by collagen, in which a size of pores on the surface of the porous powder is corresponding to a specific size of the collagen, thereby forming a collagen layer on the surface of the porous powder,

The physical cross-linking refers to an ionic cross-linking, in which collagen and chitosan have opposite charges in specific pH ranges, and thus collagen can cross-link with chitosan without using a chemical cross-linking agent.

The various bases of the present invention refer to, for example, water, a polar oil, a non-polar oil, an oil-in-water emulsion, a hydrogel and the like. Specifically, the polar oil may be, for example, triglyceride, propylene glycol isostearate, dioctyl sebacate, 4-methoxycinnamic acid 2-ethylhexyl ester, trimethylolpropane-tri-2-ethyl-hexanoate, bis(2-ethylhexyl) succinate, propylene glycol dicaptylateidicaprate, 2-ethylhexyl -cyano-3,3-diphenylacrylate, caprylic/capric triglyceride, trimethylpropane tricaprylate, tricaprylin, caprylyl glycol, dimyristoyl glycerol, bis(2-hydroxyethyl) ether dilaurate, pentaerythrityl tetrakis-(2-ethylhexanoate), glyceryl tri(2-ethylhexanoate), glycerin diacetate monostearate, octyl dodecyl lactate, propylene glycol monostearate, propylene glycol monooleate, oleyl lactate, propylene glycol dicaproate, diisopropyl sebacate, ethylene glycol monostearate, diethylene glycol sebacate, glycerol dicocoate, diluarin, glyceryl sesquioleate, ethylene glycol monooleate coconut alcohol, lauryl alcohol, cetyl lactate diethyl sebacate, sun flower fatty acid methyl ester, glycol palmitate, polyethylene glycol dilaurate, tripropylene glycol pivalate or a combination thereof.

Specifically, the non-polar oil may be, for example, silicone oil, mineral oil, vaseline, squalene, squalane, other oil having a dielectric constant less than 15 or a combination thereof.

The double-layered wrap of the present invention refers to the collagen layer covering the porous powder, and the chitosan layer physically cross-linking with the collagen layer.

The porous powder, the collagen and the chitosan used in the method of producing the composite powder are described in detailed as follows.

### Porous Powder

The porous powder of the present invention refers to an organic powder, an inorganic powder or a combination thereof having a specific surface area of 0.5 m²/₉ to 200 m²/g and an average particle size of 2 μm to 20 μm.

In one example, the aforementioned organic powder may be, for example, polymethylmethacrylate, carbon black, polyamide resin, polyethylene resin, polypropylene resin, cellulosic resin, polystyrene, styrene/acrylic acid copolymer, polysiloxane, nylon or a combination thereof. In another example, the aforementioned inorganic powder is silicon dioxide, titanium dioxide, alumina, barium sulfate, aluminium hydroxide, calcium carbonate, magnesium silicate, magnesium carbonate, aluminum silicate or a combination thereof.

Preferably, the organic powder may be polymethylmethacrylate or carbon black, and the inorganic powder may be silicon dioxide or titanium dioxide.

If the specific surface area of the porous powder was less than 0.5 m²/g, adherence of the collagen to the porous powder would be insufficient, leading to decrease in the dispersity and the moisture retention of the produced composite powder. If the average particle size of the porous powder was more than 20 μm, the produced composite powder would be likely sliding off a skin in subsequent applications.

### Collagen

The collagen of the present invention is soluble to a first solution, in which the first solution has a pH of 5 to 9. In one example, a weight ratio of the collagen in the first solution is 0.001 to 0.2.

The collagen of the present invention may have a first weight-average molecular weight of 3 kDa to 30 kDa. In a preferable example, the first weight-average molecular weight of the collagen is 3 kDa to 10 kDa. In addition, the collagen refers to, for example, a type I collagen, and preferably is type I atelocollagen.

The first solution of the present invention may be or example, water, a hydrochloric acid solution or an acetic acid solution at pH 5 to pH 9.

In one embodiment, based on an amount of the porous powder as 100 parts by weight, an amount of the collagen is 0.1 parts by weight to 20 parts by weight, and preferably is 1 part by weight to 5 parts by weight.

If the weight ratio of the collagen and the first solution was less than 0.001, or the amount of the collagen was less than 0.1 parts by weight, an adhesion percentage of the collagen to the porous powder would be lowered down during a first mixing step performed on the collagen and the porous powder, leading to decrease in the dispersity of the moisture retention of the produced composite powder. Besides, if the weight ratio was greater than 0.2, or the amount of the collagen was greater than 20 parts by weight, solubility of the collagen in the first solution would decrease.

It is noted that the physical adhesion is applied to wrap the aforementioned porous powder by collagen in the present invention. Accordingly, the present invention excludes using a common chemical cross-linking agent to improve adhesion between powders and other molecules.

### Chitosan

The chitosan of the present invention is soluble to a second solution, in which the second solution has a pH of 4 to 7. A weight ratio of the chitosan and the second solution may be 0.001 to 0.2, and preferably is 0.01 to 0.5.

A second weight-average molecular weight of the chitosan of the present invention is 3 kDa to 300 kDa, and preferably is 10 kDa to 100 kDa.

The second solution of the present invention may be the same as or different from the first solution. In one example, the second solution may be, for example, water, a lactic acid solution, an acetic acid solution, a citric acid solution, a mandelic acid solution or a hydrochloric acid solution at pH 4 to pH 7.

In one embodiment, based on the amount of the porous powder as 100 parts by weight, an amount of the chitosan is 0.1 parts by weight to 20 parts by weight, and preferably is 1 part by weight to 5 parts by weight.

If the solution had a pH of lower than 4 or greater than 7, adhesion to the collagen and solubility of the chitosan in the second solution would decrease. If the aforementioned weight ratio of the chitosan and the second solution was less than 0.001, or the amount of the chitosan was less than 0.1 parts by weight, the moisture retention and the dispersity of the produced composite powder would decrease. If the weight ratio of the chitosan and the second solution was greater than 0.2, or the amount of the chitosan was greater than 20 parts by weight, solubility of chitosan in the second solution would also decrease.

### Additive

The produced composite powder of the present invention may be combined with other additives, so as to apply the composite powder in various fields (e.g. skin care products or makeups). In one embodiment the additive may include but is not limited to a pigment, a surfactant and the like.

Specific examples of the pigment may be, for example, a red inorganic pigment such as iron oxide, iron hydroxide, iron orthotitanate a brown inorganic pigment such as γ-iron oxide and the like, a yellow inorganic pigment such as iron oxide yellow, Chinese yellow and the like, a black inorganic pigment such as iron oxide black, carbon black and the like, a violet inorganic pigment such as manganese violet, cobalt violet and the like, a green inorganic pigment such as chromium hydroxide, chromium oxide, cobalt oxide, cobalt orthotitanate and the like, a blue inorganic pigment such as iron blue, ultramarine and the like, a pigment obtained from laking a coal tar color, a pigment obtained from laking a natural pigment, a conjugated pigment obtained from conjugating powders of the above pigments and the like; a pearlescent pigment such as titanium oxide-coated mica, bismuth oxychloride, titanium oxide-coated bismuth oxychloride, titanium oxide-coated talc, fish scale flake, titanium oxide-coated colored mica and the like; a metal powder pigment such as aluminum powder, copper powder, stainless steel powder and the like; a coal tar colors such as Red No 3, Red No. 104, Red No. 106, Red No 201, Red No. 202, Red No. 204, Red No. 205, Red No. 220, Red No. 226, Red No. 227, Red No. 228, Red 230 No. , Red No. 401, Red No. 505, Yellow No. 4, Yellow No. 5, Yellow No. 202, Yellow No. 203, Yellow No. 204, Yellow No. 401, Blue No. 1, Blue No. 2, Blue No. 201, Blue No. 404, Green No. 3, Green No. 201, Green. No. 204, Green No. 205, Orange No. 201, Orange No. 203, Orange No. 204, Orange No. 206, Orange No. 207 and the like; a natural pigment such as carmine acid, laccaic acid, cartharnin, brazilin, crocin and the like.

The aforementioned surfactant may be one of a cationic surfactant, a non-ionic surfactant or an anionic surfactant.

Based on the amount of the porous powder is 100 parts by weight, an amount of the additive may be, for example, 0.1 parts by weigh to 20 parts by weight.

### Preparation of Composite Powder

In the method of producing the composite powder of the present invention, a first mixing step is firstly performed on the porous powder and the collagen, so as to form a first mixture solution. In one embodiment, the first mixing step is performed at a first temperature of 4° C. to 40° C. for 5 minutes to 2 hours.

The first mixing step can be, for example, performed by a stirring equipment to sufficiently mix the collagen and the porous powder. There is no special limitation to the stirring equipment, as long as the porous powder can be completely dispersed and adhered by the collagen.

In the first mixing step, the collagen having a specific size physically adheres to pores on the surface of the porous powder, thereby forming the collagen layer on the surface of the porous powder. Therefore, the first mixture solution may include the collagen not adhering to the porous powder, the porous powder, the first solution and the porous powder adhered by the collagen.

Therefore, if the first temperature was higher than 40° C., the collagen would be likely denatured and lost its original function. In addition, if the first mixing step was performed for less than 5 minutes, the physical adhesion would be incomplete, leading to low adhesion percentage of the collagen to the surface of the porous powder. However, if the first mixing step was performed more than 2 hours, the adhesion percentage would not increase anymore and a time cost would relatively increase.

Next, a first filtering step is performed on the first mixture solution, so as to form the semifinished powder. The semifinished powder refers to the porous powder adhered by the collagen. For example, the first filtering step may be performed by centrifuge in a rate of 3000 rpm, so as to precipitate the semifinished powder on the bottom and separate the semifinished powder from the collagen not adhering to the porous powder, the porous powder and the first solution. However, other methods such as a column chromatography separation technique may be applied without any special limitation of the present invention.

Afterwards, a second mixing step is performed on the chitosan and the aforementioned semifinished powder in the second solution, so as to form a second mixture solution. In one embodiment, the second mixing step may be performed at a second temperature of 4° C. to 40° C. for 5 minutes to 2 hours. In the second mixing step, an amino group of the chitosan bears a positive charge due to the second solution at pH 5 to pH 7. On the other hand, a carboxyl group of the collagen on the porous powder bears a negative charge. By the opposite positive charge and negative charge, the chitosan and the collagen ionically cross-link to each other (i.e. physically cross-link), and the chitosan is connected to the porous powder through the collagen, thereby forming a chitosan layer on the collagen layer. Therefore, the second mixture solution includes the un-crosslinked chitosan, the second solution, the semifinished powder and the semifinished powder wrapped by the chitosan (i.e. the composite powder).

If the second mixing step was performed at a temperature lower than 4° C. or for less than 5 minutes, the physical cross-linking effect between the chitosan and the collagen would be not sufficient, and thus an amount of the chitosan on the porous powder might be too low to achieve sufficient dispersity and moisture retention. On the other hand, if the second mixing step was performed at a temperature higher than 40° C., the collagen in the second mixture solution would be likely decomposed or denatured.

Then, the second mixture solution is subjected to a second filtering step. The composite powder is separated from the un-crosslinked chitosan, the second solution and the semifinished powder. The second filtering step can be performed by a similar method to the first filtering step rather than further described here.

In one example, a drying step may be performed after the second filtering step, so as to further dry the composite powder for better storage and application afterwards. There is no special limitation to the drying step of the present invention, and the drying step may be, for example, a cool drying step, a baking step, a vacuum dehydration step and the like. It is noted that a temperature of the baking step is not higher than 50° C. to avoid denaturation and decomposition of the collagen.

In another embodiment, additional additives may be further added after the composite powder is formed, so as to apply the composite powder to fields of makeups, skin care products, pharmaceutical products and the like. The specific examples of the additives are described as above,

It is noted that if the collagen and the chitosan were premixed or simultaneously added into the porous powder, the cross-linking reaction between the collagen and the chitosan would occur firstly due to a better reactivity of the collagen and the chitosan, thereby forming a molecule having a large volume which fails to adhere to the porous powder. On the other hand, if the chitosan adhered directly to the porous powder, a linkage between the chitosan and the collagen is weak and the collagen would be probably peeled from the porous powder.

### Composite Powder

The composite powder produced by the aforementioned method has an average particle size of 3 μm to 25 μm. To be specific, the produced composite powder takes the porous powder as a base, the collagen layer wraps the surface of the porous powder, and the chitosan layer wraps the collagen layer. With the moisture retention and bio compatibility of collagen and chitosan, the produced composite powder is able to increase the moisture retention and the bio compatibility (skin fitness) of the application products. Furthermore, as collagen and chitosan are polar molecules, the composite powder of the present invention has good dispersity in bases such as water, the polar oil, the oil-in-water emulsion, the hydrogel and the like. Moreover, hydrophlic collagen and chitosan decrease greasiness of the composite powder.

It is noted that the collagen layer and the chitosan layer formed on the porous powder mainly fills the pores on the porous powder, while not substantially affect the average particle size of the porous powder. Therefore, the average particle size of the composite powder is substantially same as or similar to the average particle size of the porous powder.

### Oil-in-Water Cosmetic Composition

The composite powder of the present invention may be applied to produce the oil-in-water cosmetic composition. The oil-in-water cosmetic composition includes an oil-in-water emulsion base and the aforementioned composite powder uniformly dispersed therein. In one example, the emulsion may be lotion or cream.

### Hydrogel Cosmetic Composition

The composite powder of the present invention may be also applied to the hydrogel cosmetic composition. The hydrogel cosmetic composition includes a hydrogel base and the composite powder uniformly dispersed therein. In one example, the hydrogel base may be, for example, cellulose ether (e.g. hydroxyethyl cellulose, methyl cellulose, hydroxypropyl methyl cellulose), polyvinylpyrrolidone, polyvinyl alcohol), guar gum, hydroxypropyl guar gum and xanthan gum.

A few examples are used to describe a specific flow of the method of producing the composite powder and evaluation results of the produced composite powder.

### EXAMPLE 1

100 parts by weight of porous sphere particles of polyrnethylmethacrylate (an average particle size of 8 μm; a specific surface area of 85 m²/g; a trade name of SUNPMMA-S; produced by Sunjin Chemical Co. Ltd) and 5 parts by weight of type 1 collagen (weight-average molecular weight of 3 kDa) dissolved in 0.01 M of an acetic acid solution were disposed in a stirrer (trade name: Rw28basic; produced by IKA company), and stirred at a rate of 100 rpm for 20 minutes under 25° C. to form the first mixing solution. Next, the first mixing solution was subjected to a filtering step in a centrifuge at rate of 3000 rpm, so as to precipitate and separate the semifinished powder. Then, 3 parts by weight of chitosan (weight-average molecular weight of 100 kDa) dissolved in 0.0001M of a dilute hydrochloric acid solution was added into the semifinished powder and stirred by the aforementioned stirrer to sufficiently ax the semifinished powder and chitosan to form the second mixture solution. Next, the second mixture solution was subjected to the precipitating and filtering step by centrifuge at a rate of 3000 rpm, thereby forming the composite powder. The compositions and evaluation results of Example 1 are shown as Table 1.

### EXAMPLE 2 TO EXAMPLE 4

Example 2 to Example 4 were performed by the same method as Example 1, while the species or the amounts of the compositions were changed. The species and the amounts of the compositions, and evaluation results of Example 2 to Example 4 are shown as Table 1 rather than further described here.

### Comparison Example 1 to Comparison Example 4

The porous powder of Comparison Example 1 to Comparison Example 4 were directly subjected to evaluation without wrapped by collagen and chitosan. The species of the porous powder and evaluation results of Comparison Example 1 to Comparison Example 4 are shown as Table 1.

### Comparison Example 5 and Comparison Example 6

Comparison Example 5 and Comparison Example 6 were respectively wrapping the porous only by collagen or chitosan, and were subjected to the evaluation. The species of the porous powder and the evaluation results of Comparison Example 5 and Comparison Example 6 are shown as Table 1.

### Evaluation

### 1. Dispersity

The dispersity of the composite powder produced by the method of the present invention is respectively evaluated in common bases such as water, the polar oil, the oil-in-water cream and the hydrogel. Situations such as lamination, aggregation are evaluated.

### 1-1 Dispersity in Water

The dispersity in water of the present invention refers to a degree of dispersion when the composite powder is added into deionized water. The dispersity in water is evaluated by uniformly mixing the composite powder and the deionized water with a weight ratio of 1:20 in a test tube and the mixture is left standing for a while. Time for the composite powder precipitating to the bottom of the test tube is then observed. The evaluation standards are shown as follows, in which the longer the time for the composite powder precipitating to the bottom of the test tube, the better the dispersity in water.

◯: the time for the composite powder precipitating to the bottom of the test tube is more than 30 minutes.

Δ: the time for the composite powder precipitating to the bottom of the test tube is 5 minutes to 30 minutes.

X: the time for the composite powder precipitating to the bottom of the test tube is less than 5 minutes.

### 1-2 Dispersity in Polar Oil

The dispersity in a polar oil (i.e. caprylic/capric triglyceride) of the present invention refers to a degree of dispersion when the composite powder is added into the polar oil. The dispersity in the polar oil is evaluated by uniformly mixing the composite powder and the polar oil with a weight ratio of 1:20 in a test tube and the mixture is left standing for a while. Time for the composite powder precipitating to the bottom of the test tube is then observed. The evaluation standards are shown as follows, in which the longer the time for the composite powder precipitating to the bottom of the test tube, the better the dispersity in polar oil.

◯: the composite powder precipitating to the bottom of the test tube is precipitated in a period of more than 30 minutes.

Δ: the composite powder precipitating to the bottom of the test tube is precipitated in a period of 5 minutes to 30 minutes.

X: the composite powder precipitating to the bottom of the test tube is precipitated in a period of less than 5 minutes.

### 1-3 Stability in Oil-in-Water Cream

The stability in oil-in-water cream refers to the dispersity of the composite powder after it is added into the oil-in-water cream (i.e. aggregation or lamination). The stability in oil-in-water cream oil is evaluated by uniformly mixing the composite powder and the oil-in-water cream with a weight ratio of 1:20 in a test tube and directly observing the lamination of the mixture. The evaluation standards are as follows, in which the less apparent the lamination, the better the stability in the oil-in-water cream.

◯: The lamination is not apparent.

Δ: An emulsion layer exists.

X: The lamination is apparent.

### 1-4 Mixing Uniformity in Hydrogel

The mixing uniformity in the hydrogel of the present invention refers to observing whether aggregation occurs when the composite powder is mixed with the hydrogel. The mixing uniformity in the hydrogel is evaluated by mixing the composite powder and the hydrogel with a weight ratio of 1:20, and directly observing whether aggregates formed in the mixture. The less aggregation, the better the mixing uniformity, indicating that the composite powder is applicable to a hydrogel-based product. The evaluation standards are shown as follows:

◯: The aggregation is not apparent.

Δ: Aggregated particles exist.

X: The aggregation is apparent.

### Moisture Retention

The moisture retention of the present invention is evaluated by a sensory test which the composite powder is coated on a human skin. The evaluation standards are shown as follows.

◯: good moisture retention.

Δ: proper moisture retention.

X: insufficient moisture, retention.

### 3. Greasiness

The greasiness of the present invention is directly evaluated by a sensory test which the composite powder is coated on the human skin to evaluate the composite powder is greasy or not. The evaluation standards are shown as follows:

◯: not greasy.

Δ: a little greasy.

X: quite greasy.

According to Example to Example 4 of Table 1, the composite powder formed by the method of the present invention has good dispersity in various common bases (water, the polar oil, the oil-in-water cream and the hydrogel) used in makeups or skin care products, and has sufficient moisture retention and low greasiness. However, according to Comparison Example 1 to Comparison Example 4, if the porous powder was not physically adhered by the collagen and physically cross-linked by the chitosan, the desired dispersity, moisture retention and low greasiness could not be achieved. In addition, according to Comparison Example 5 and Comparison Example 6, if the porous powder was only wrapped by a single layer of the collagen layer or the chitosan layer, the desired dispersity, moisture retention and low greasiness could not be achieved, either.

The composite powder having an average particle size of 3 μm to 25 μm is produced by applying the composite powder and the method of producing the same of the present invention. The composite powder may have a double-layered wrap of the collagen layer and the chitosan layer, and thus the composite powder can have good dispersity, high moisture retention and low greasiness. In addition, the composite powder may be applied to makeups, skin care products or pharmaceutical products by adding additional additives.

TABLE 1

| | | | Example | | | | Comparison Example | | | | | |
|---|---|---|---|---|---|---|---|---|---|---|---|---|
| | | | 1 | 2 | 3 | 4 | 1 | 2 | 3 | 4 | 5 | 6 |
| Composite | Porous | A-1 | 100 | — | — | — | 100 | — | — | — | 100 | — |
| powder | powder | A-2 | — | 100 | — | — | — | 100 | — | — | — | 100 |
| composition | | A-3 | — | — | 100 | — | — | — | 100 | — | — | — |
| (part by | | A-4 | — | — | — | 100 | — | — | — | 100 | — | — |
| weight) | Collagen | B-1 | 5 | — | 5 | — | — | — | — | — | 5 | — |
| | | B-2 | — | 3 | — | 3 | — | — | — | — | — | — |
| | Chitosan | C-1 | 3 | — | 3 | — | — | — | — | — | — | 5 |
| | | C-2 | — | 5 | — | 5 | — | — | — | — | — | — |
| | First | D-1 | 100 | 100 | 100 | 100 | — | — | — | — | 100 | — |
| | solution | | | | | | | | | | | |
| | Second | E-1 | 100 | 100 | 100 | 100 | — | — | — | — | — | 100 |
| | solution | | | | | | | | | | | |
| Composite | Average particle size (μm) | | 8 | 5 | 3 | 8 | — | — | — | — | 8 | 5 |
| powder | | | | | | | | | | | | |
| Evaluation | Dispersity | Dispersity in water | Δ | ◯ | Δ | ◯ | X | Δ | X | Δ | X | Δ |
| method | | Dispersity in polar oil | ◯ | Δ | ◯ | Δ | ◯ | X | Δ | Δ | ◯ | X |
| | | Stability in oil-in-water cream | ◯ | ◯ | ◯ | ◯ | Δ | Δ | Δ | Δ | Δ | Δ |
| | | Mixing uniformity in hydrogel | ◯ | ◯ | ◯ | ◯ | Δ | Δ | Δ | Δ | Δ | Δ |

| | | | | | | | | | | |
|---|---|---|---|---|---|---|---|---|---|---|
| Moisture retention | Δ | ◯ | Δ | ◯ | X | Δ | X | Δ | X | Δ |
| Greasiness | ◯ | ◯ | ◯ | ◯ | Δ | Δ | Δ | Δ | Δ | ◯ |

A-1 polymethylmethacrylate (average particle size: 8 μm, specific surface area: 85 m²/g: trade name; SUNPMMA-S; produced by Sunjin)
A-2 silicon dioxide (trade name: SUNJIN, average particle size: 5 μm; produced by WWRC TAIWAN Co., Ltd.)
A-3 carbon black (average particle size: 3 μm; produced by FuRui Chemical Industry Co., LTD.)
A-4 titanium dioxide (reagent grade titanium oxide, 8 average particle size: 8 μm; produced by First Chemical Manufacture Co., Ltd.)
B-1 collagen (weight-average molecular weight: 3 kDa)
B-2 collagen (weight-average molecular weight: 30 kDa)
C-1 chitosan (weight-average molecular weight: 100 kDa)
C-2 chitosan (weight-average molecular weight: 10 kDa)
D-1 0.01M acetic acid solution, about pH 5
E-1 0.0001M dilute hydrochloric acid solution, about pH 4

What is claimed is:

1. A method of producing a composite powder, comprising:
performing a first mixing step on a porous powder and collagen in a first solution, so as to form a first mixture solution, wherein the collagen has a first weight-average molecular weight of 3 kDa to 30 kDa;
performing a first filtering step on the first mixture solution, so as to form a semifinished powder;
performing a second mixing step on chitosan and the semifinished powder in a second solution, so as to form a second mixture solution, wherein the chitosan has a second weight-average molecular weight of 3 kDa to 300 kDa; and
performing a second filtering step on the second mixture solution, thereby forming the composite powder, and
wherein based on an amount of the porous powder as 100 parts by weight, an amount of the collagen is 0.1 parts by weight to 20 parts by weight, and an amount of the chitosan is 0.1 parts by weight to 20 parts by weight.

2. The method of claim 1, wherein the porous powder comprises an organic powder, an inorganic powder or a combination thereof having a specific surface area of 0.5 m²/g to 200 m²/g and a first average particle size of 2 μm to 20 μm.

3. The method of claim 1, wherein the first solution has a pH of 5 to 9, and the second solution has a pH of 4 to 7.

4. The method of claim 1, wherein a weight ratio of the collagen and the first solution is 0.001 to 0.2, and a weight ratio of the chitosan and the second solution is 0.001 to 0.2.

5. The method of claim 1, wherein the first mixing step is performed at a first temperature of 4° C. to 40° C. for 5 minutes to 2 hours.

6. The method of claim 1, wherein the second mixing step is performed at a second temperature of 4° C. to 40° C. for 5 minutes to 2 hours.

7. The method of claim 1, wherein the first solution comprises water, an acetic acid solution or a hydrochloric acid solution, and the second solution comprises water, a lactic acid solution, an acetic acid solution, a citric acid solution, a mandelic acid solution or a hydrochloric acid solution.

8. A composite powder, obtained by a method of claim 1, wherein the composite powder comprises:
a porous powder;
a collagen layer, covering an outer surface of he porous powder; and
a chitosan layer, covering the collagen layer.

9. The composite powder of claim 8, wherein a second average particle size of the composite powder is 3 μm to 25 μm.

10. An oil-in-water cosmetic composition comprising an oil-in-water emulsion base and the composite powder of claim 8 that is uniformly dispersed therein.

11. A hydrogel cosmetic composition comprising a hydrogel base and the composite powder of claim 8 that is uniformly dispersed therein.
